(19) United States
(12) Patent Application Publication
(10) Pub. No.: US 2019/0307385 A1
(43) Pub. Date: Oct. 10, 2019

(54) SYSTEMS AND METHODS FOR ASSESSMENT OF COGNITIVE STATE

(71) Applicant: HONEYWELL INTERNATIONAL INC., Morris Plains, NJ (US)

(72) Inventors: Zdenek Moravek, Rozdrojovice (CZ); Pavel Badin, Dolni Kounice (CZ)

(73) Assignee: HONEYWELL INTERNATIONAL INC., Morris Plains, NJ (US)

(21) Appl. No.: 15/949,615

(22) Filed: Apr. 10, 2018

(51) Int. Cl. A61B 5/16; A61B 5/18

(52) U.S. Cl. CPC A61B 5/165; G08B 21/06; A61B 5/168; A61B 5/18

(57) ABSTRACT

Methods and systems for classifying a subject's cognitive state are provided. The method receives and stores sensor signals comprising a first sensor signal (SS_1) from a first sensor configured to sense a first aspect of the subject, and a second sensor signal (SS_2) from a second sensor configured to sense a second aspect of the subject. The validity of SS_1 and SS_2 are determined using a binary signal validity methodology and a weighted signal validity methodology. SS_1 and SS_2 are analyzed to identify patterns therein, and the patterns are defined as either an objective marker or a subjective marker. A subject profile is referenced and updated through multiple iterations. The subject's cognitive state is subsequently classified based on both adjusted subjective and objective markers.

### SYSTEMS AND METHODS FOR ASSESSMENT OF COGNITIVE STATE GOVERNMENT RIGHTS

The project leading to this application has received funding from the Clean Sky 2 Joint Undertaking under the European Union's Horizon 2020 research and innovation programme under grant agreement NO CS2-LPA-GAM-2014-2015-1.

### TECHNICAL FIELD

The present invention generally relates to cognitive state assessment systems, and more particularly relates to methods and systems that assess a subject's cognitive state.

### BACKGROUND

As a subject's cognitive state declines, his ability to perform his job function, operate equipment, and make decisions may be adversely affected. From a project management perspective, this may translate into reduced efficiency and reduced accuracy, as well as potentially preventing successful completion of a task.

Accordingly, tools that capably assess cognitive state are desirable. Furthermore, other desirable features and characteristics of the present disclosure will become apparent from the subsequent Detailed Description and the appended claims, taken in conjunction with the accompanying drawings and this Background.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

Provided is a method for classifying a subject's cognitive state. The method comprises: at a control module, for a predetermined amount of time (epoch), continuously: receiving and storing sensor signals comprising a first sensor signal (SS_1) from a first sensor configured to sense a first aspect of the subject, and a second sensor signal (SS_2) from a second sensor configured to sense a second aspect of the subject; determining that SS_1 is valid, based on human physiology models; determining that SS_2 is valid, based on the human physiology models; analyzing SS_1 to identify a pattern therein, the pattern defined as an objective marker; and sequentially performing the steps of, (a) assigning a binary state classification based on the objective marker; and (b) analyzing SS_2 to identify a pattern therein, the pattern defined as a subjective marker; (c) creating an association between the binary state classification and the subjective marker; (d) identifying a baseline parameter for the subjective marker, the baseline parameter being unique for the subject; (e) transforming the subjective marker to a second objective marker using adaptive data filtration and the baseline parameter; and (f) classifying the subject's cognitive state using the first objective marker and the second objective marker.

A system for assessment of cognitive state of a subject is provided. The system comprises: a source of sensor signals associated with the subject; a state regulator configured to receive a binary cognitive state and to generate therefrom commands for a user interface; and a control module comprising human physiology models, the control module configured to: receive a first sensor signal (SS_1) and a second sensor signal (SS_2); determine that SS_1 is valid with a binary validity test; determine that SS_2 is valid with a binary validity test; analyze SS_1 to identify a pattern therein, the pattern defined as an objective marker; and sequentially perform the steps of, (a) assign a binary state classification based on the objective marker; and (b) analyze SS_2 to identify a pattern therein, the pattern defined as a subjective marker; (c) create an association between the binary state classification and the subjective marker; (d) identify a baseline parameter for the subjective marker, the baseline parameter being unique for the subject; (e) transform the subjective marker to a second objective marker using adaptive data filtration and the baseline parameter; and (f) classify the subject's cognitive state using the first objective marker and the second objective marker.

Also provided is another method for classifying a subject's cognitive state. The method comprises: at a control module, continuously: receiving and storing sensor signals comprising a first sensor signal (SS_1) from a first sensor configured to sense a first aspect of the subject, and a second sensor signal (SS_2) from a second sensor configured to sense a second aspect of the subject; analyzing SS_1 to identify a pattern therein, the pattern defined as an objective marker; assigning a binary state classification based on the objective marker; analyzing SS_2 to identify a pattern therein, the pattern defined as a subjective marker; cross validating SS_1 with SS_2 to thereby (1) determine that SS_1 is valid, (2) determine that SS_2 is valid, and (3) assign (i) a first weight to SS_1, and (ii) a second weight to SS_2; creating an association between the binary state classification and the subjective marker; identifying a baseline parameter for the subjective marker, the baseline parameter being unique for the subject; transforming the subjective marker to a second objective marker using adaptive data filtration and the baseline parameter; and classifying the subject's cognitive state using the first objective marker and the second objective marker.

Furthermore, other desirable features and characteristics of the system and method will become apparent from the subsequent detailed description and the appended claims, taken in conjunction with the accompanying drawings and the preceding background.

### BRIEF DESCRIPTION OF THE DRAWINGS

The present invention will hereinafter be described in conjunction with the following drawing figures, wherein like numerals denote like elements, and

FIG. 1 is a block diagram of system for assessment of cognitive state, in accordance with an exemplary embodiment;

FIG. 2 is a block diagram of a control module for system for assessment of cognitive state, in accordance with an exemplary embodiment;

FIGS. 3-5 are a simplified flow chart for a method for assessment of cognitive state, in accordance with an exemplary embodiment; and

FIG. 6 is a system diagram providing more detail for a system and method for assessment of cognitive state, in accordance with an exemplary embodiment.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the invention and not to limit the scope of the invention that is defined by the claims. Furthermore, there is no intention to be bound by any theory presented in the preceding background or the following detailed description.

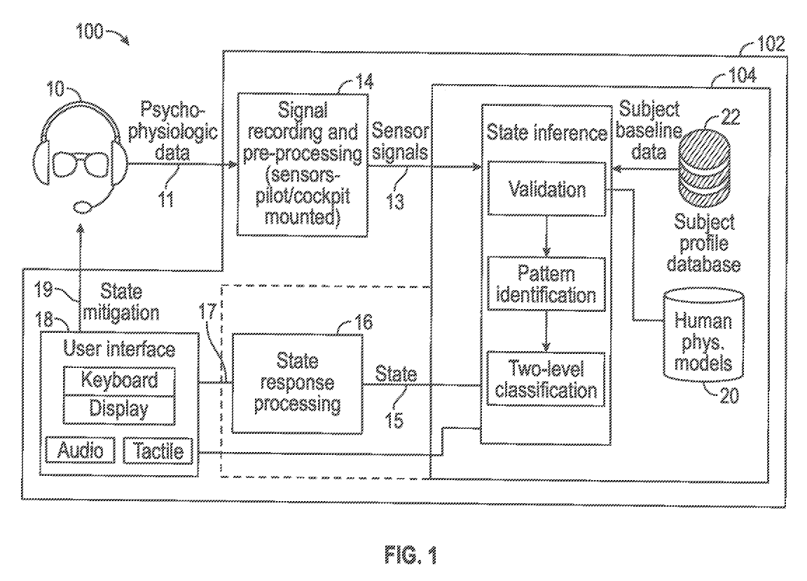

Exemplary embodiments of the novel disclosed system provide a technologically improved system and method for real time assessment of cognitive state (FIG. 1, 102). As used herein, “real-time” is interchangeable with current and instantaneous. A subject's (FIG. 1, 10) cognitive state is inferred by measuring and interpreting psycho-physiologic data (also referred to herein as biometric data FIG. 1, 11). The system for assessment of cognitive state 102 senses, pre-processes, and records the biometric data 11. As used herein, “recording” means storing data in a data storage location referred to as a buffer, and received sensor signals are recorded in real-time, as received. Within the system for assessment of cognitive state 102, a control module for assessment of cognitive state (FIG. 1, 104) receives the processed biometric data 11 as sensor signals (FIG. 1, 13), and processes them in accordance with an algorithm and novel set of rules described hereinbelow, to determine therefrom a binary cognitive state classification for the subject 10. The binary cognitive state classification for the subject 10 is determined in real-time. The disclosed system for assessment of cognitive state 102 and methods are described in more detail below.

Several terms and phrases are repeatedly used herein. In order to provide context for these terms and phrases, the following definitions and examples are provided below:

Cognitive state: A subject's 10 state of mind. Examples of a subject's cognitive state include, without limitation: awake, asleep, agitated, and at rest. A binary cognitive state is one that is either true or false; for example, a person “is” awake (i.e., awake=true) or “is not” awake (i.e., awake=false).

Aspect: An externally manifested and individually measurable (i.e., sense-able) characteristic of a given cognitive state. Some non-limiting examples of aspects include: the electrical signals of the heart, respiration, pressure of a hand on a user input device, body weight distribution in a chair, head direction, head movement, perspiration, eyelid position, pupil diameter, etc. The biometric data 11 is the measurable component of the aspect. In the herein described system for assessment of cognitive state 102, for each aspect, at least one appropriately configured sensor is oriented to sense the aspect (as biometric data 11) and to generate therefrom a respective sensor signal 13. As may be appreciated, for any given cognitive state, each biometric data 11 (and respective sensor signal 13) may vary from one subject 10 to the next. For example, a range of electrical signals measured from the heart of an athletic subject during awake state may vary from a range of electrical signals measured from the heart of a sedentary subject during awake state.

Objective marker: A first kind of detectible pattern in a sensor signal 13. Each sensor signal 13 may be analyzed to identify features or patterns therein. Objective markers, as used herein, refer to identified patterns that are substantially the same for all subjects. An example objective marker is “eyes open,” identified from a sensor signal 13 for eyelid position biometric data 11. Based on an objective marker, a cognitive state may be assigned (i.e., the cognitive state “awake” may be assigned to “eyes open”).

Subjective marker: Another kind of detectible pattern in a sensor signal 13. As used herein, subjective markers refer to features or patterns that can be identified in a sensor signal 13 that tend to vary from a first subject to a second subject (perhaps with a degree of overlap between subjects). An example of a subjective marker is heart rate, identified from a sensor signal for electrical signals from the heart. As may be readily appreciated, for a cognitive state, such as awake, the heart rate of a subject generally has a range, and, from one subject to another subject, the range of heart rate while awake may vary (for example, as a function of age, fitness, anxiety, medications, etc.).

Epoch: Identifying a pattern in a sensor signal 13 implies that the sensor signal 13 be monitored for a certain duration of time sufficient to identify a pattern. For example, the electrical signals produced by the heart are monitored for a period of time sufficient to identify a heart rate, and an eyelid position is monitored for a period of time to distinguish between a quick blink (consistent with someone awake) and the eyes being closed (consistent with someone being asleep or unconscious). As used herein, the duration of time used is a configurable, predetermined, amount of time referred to as an “epoch.”

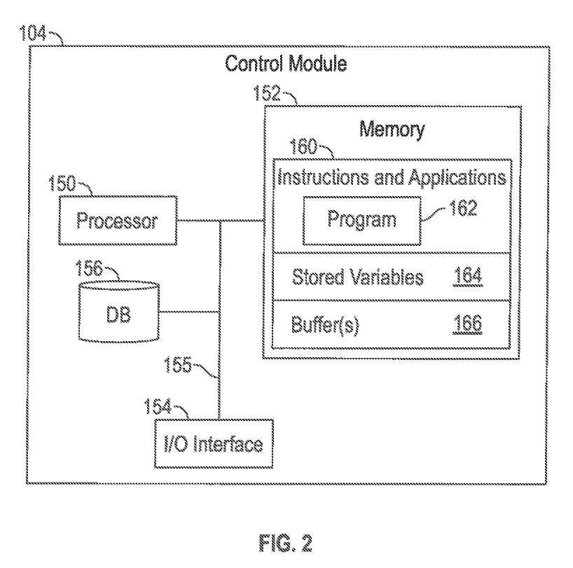

As may be appreciated based on the above description, assigning a binary state classification, such as “awake,” based on an objective marker has a high reliability. In contrast, assigning a binary state classification, such as “awake,” based solely on a subjective marker, such as “heart rate,” may have a low reliability. A technological improvement provided by the control module 104 is the development and continuous improvement of a “subject profile” for a specific subject, via many iterations of the novel algorithm (FIG. 2, program 162) described herein. A technological effect of the control module 104 is the ability to use a subject profile for a given subject 10 in the validation of other contemporaneously received sensor signals 13, and in the assessment of the subject's cognitive state. The objective and subjective markers may be processed to recognize a change in cognitive state, such as the deterioration of a subject's cognitive state. From there, actuators in various components of a user interface (FIG. 1, 18), may be utilized to alert the subject 10.

Turning now to FIGS. 1 and 2, in an embodiment, the system for assessment of cognitive state 102 (also referred to herein as “system” 102) may be separate from, or integrated within, a preexisting mobile platform management system, avionics system, cockpit display system (CDS), flight controls system (FCS), aircraft flight management system (FMS), or electronic flight bag (EFB). The system 102 may comprise, in various embodiments, the control module for assessment of cognitive state 104 (also referred to herein as “control module” 104) operatively coupled to one or more of: a signal recording and pre-processing system 14, a state regulator 16 and a user interface 18. These functional blocks, and their interaction, are described in more detail below.

The signal recording and pre-processing system 14 is shown as one functional block, but in practice, it may be multiple, variously located sensors and their corresponding transducers. As may be readily appreciated, different sensors may be employed to sense different aspects. Some sensors may be attached to a subject, such as a pilot, and some may be attached to equipment around the subject, such as a pressure sensor on a touch sensitive screen. The sensed biometric data may be of low amplitude and subject to background noise. The signal recording and pre-processing system 14 may perform signal processing methods to amplify signals and to remove artifacts and noise from individual biometric data 11 in the generation of sensor signals 13 before transmitting the sensor signals 13 to the control module 104.

The state regulator 16 performs state response processing, meaning that it receives and processes a binary cognitive state 15 from the control module 104 to determine what state mitigation 19 should occur responsive to the binary cognitive state 15. The state regulator 16 commands various components of the user interface 18 based on having determined what state mitigation should 19 occur. For example, if the state has been determined to be “asleep,” and the state regulator 16 determines that the state mitigation 19 includes emitting an audible alert from an audio device and vibrating a tactile transducer in a seat, it commands the audio component and the tactile transducer. The state regulator 16 may also generate commands to display warnings on a display. In some embodiments, the state regulator 16 functionality is integrated within the control module 104, such that the control module 104 generates the commands 17 for various components of the user interface 18 to generate or render cognitive state mitigating feedback for the subject 10.

The user interface 18 is a functional block that includes components that receive user input and components that provide output to a user. Accordingly, the user interface may include components, such as: a keypad, touchpad, keyboard, mouse, touch panel (or touchscreen), joystick, knob, line select key or another suitable device adapted to receive input from a user. The user interface may also include a display system, an audio system, a tactile transducer, a gesture controller, a speech to text component, and the like. The user interface 18 and the control module 104 are cooperatively configured to allow a user (e.g., a subject 10, a pilot, a co-pilot, or a crew member) to interact individually with each component of the user interface 18. The user interface is configured to render cognitive state mitigating feedback (state mitigation 19) using one or more of its components responsive to commands 17.

The control module 104 performs the state inference functions of the system 102. During operation, the control module 104 continuously processes sensor signals 13, and determines and transmits a binary cognitive state 15 classification for the subject 10. The functionality of the control module 104 includes (i) sensor signal validation, (ii) pattern identification, and (iii) cognitive state classification. As mentioned, in some embodiments, the control module 104 performs state response processing and generates commands 17 for the user interface 18.

The control module 104 is a module. As used herein, the term “module” refers to any hardware, software, firmware, electronic control component, processing logic, and/or processor device, individually or in any combination, configured as a means for facilitating communications and/or interaction between the elements of the system 102 and performing additional processes, tasks and/or functions to support operation of the system 102, as described herein. Depending on the embodiment, the control module 104 may be implemented or realized with a general purpose processor (shared, dedicated, or group) controller, microprocessor, or microcontroller, and memory that executes one or more software or firmware programs; a content addressable memory; a digital signal processor; an application specific integrated circuit (ASIC), a field programmable gate array (FPGA); any suitable programmable logic device; combinational logic circuit including discrete gates or transistor logic; discrete hardware components and memory devices; and/or any combination thereof, designed to perform the functions described herein.

In the control module 104 embodiment depicted in FIG. 2, a processor 150 and a memory 152 form a novel processing engine or unit that performs the processing activities of the control module 104. The processor 150 may comprise any type of processor or multiple processors, single integrated circuits such as a microprocessor, or any suitable number of integrated circuit devices and/or circuit boards working in cooperation to carry out the described operations, tasks, and functions by manipulating electrical signals representing data bits at memory locations in the system memory, as well as other processing of signals. The memory 152 is a data storage element that maintains data bits and may be utilized by the processor 150 as storage and/or a scratch pad. The memory 152 may be located on and/or co-located on the same computer chip as the processor 150.

In the depicted embodiment, the memory 152 stores instructions and applications 160 and one or more configurable variables in stored variables 164. Buffer 166 represents data storage for storing sensor signals 13 as described herein. Information in the memory 152 may be organized and/or imported from an external data source during an initialization step of a process; it may also be programmed via the user interface 18. During operation, the control module 104 references human physiology models 20 and a subject profile database 22, each of which may be memory intensive. Therefore, some embodiments of the control module may store the human physiology models 20 and the subject profile database 22 in the optional data storage element or database 156.

A novel algorithm, program 162, is embodied in the memory 152 (e.g., RAM memory, ROM memory, flash memory, registers, a hard disk, or the like) or another suitable non-transitory short or long term storage media capable of storing computer-executable programming instructions or other data for execution. The program 162 includes rules and instructions which, when executed, cause the system for assessment of cognitive state 102 to perform the functions, techniques, and processing tasks associated with the operation of the system for assessment of cognitive state 102 described herein.

During operation, the processor 150 loads and executes one or more programs, algorithms and rules embodied as instructions and applications 160 contained within the memory 152 and, as such, controls the general operation of the control module 104 as well as the system 102. In executing the process described herein, the processor 150 specifically loads and executes the instructions embodied in the program 162. Additionally, the processor 150 is configured to, in accordance with the program 162: process received inputs (from the sensor signals 13 and from the user interface 18); optionally reference the database 156; perform the processing activities described herein; and, transmit a binary cognitive state classification (state 15).

In various embodiments, the processor/memory unit of the control module 104 may be communicatively coupled (via a bus 155) to an input/output (I/O) interface 154, and the database 156. The bus 155 serves to transmit programs, data, status and other information or signals between the various components of the control module 104. The bus 155 can be any suitable physical or logical means of connecting computer systems and components. This includes, but is not limited to, direct hard-wired connections, fiber optics, infrared and wireless bus technologies.

The I/O interface 154 enables communications within the control module 104, as well as between the control module 104 and (i) other system 102 components, and (ii) external data sources not already addressed herein. The I/O interface 154 can include one or more network interfaces to communicate with other systems or components. The I/O interface 154 can be implemented using any suitable method and apparatus. For example, the I/O interface 154 supports communication from a system driver and/or another computer system. The I/O interface 154 may also include one or more network interfaces to communicate with technicians, and/or one or more storage interfaces for direct connection to storage apparatuses, such as the database 156.

The control module 104 employs a human physiology models 20 library and a subject profile database 22. As mentioned, these may be stored in memory 152 or may be stored in the optional database 156. Each of these provides information that the control module uses to determine validity of sensor signals 13. The human physiology models are rules and models with broad and all- inclusive ranges and threshold expectations for respective biometric data 11. They basically provide a rule of thumb “reality check,” as a first level of validation by the control module 104. For example, a normal resting respiratory rate is a range between 12 and 20 breaths per minute, so a sensor signal 13 that is within that range passes a first level validation. In another example, an inter-beat interval in electrical signals from a heart of a healthy person is limited to a range of 300 to 2000 milliseconds (ms); so a sensor signal 13 outside of the range is considered an artifact, or fails the first level of validation.

In contrast, the subject profile database 22 stores, for a specific subject 10, his or her unique ranges and threshold expectations for the biometric data 11. For example, a first subject 10 may have a resting respiratory rate of 12-15 breaths per minute, and resting inter-beat interval of 1800-2000 ms. As the control module 104 performs multiple iterations on the first subject 10, the subject baselines for the first subject 10 become more accurate. As a technological advantage, the resulting increased accuracy enables the use of individual subjective markers to identify the first subject's cognitive state.

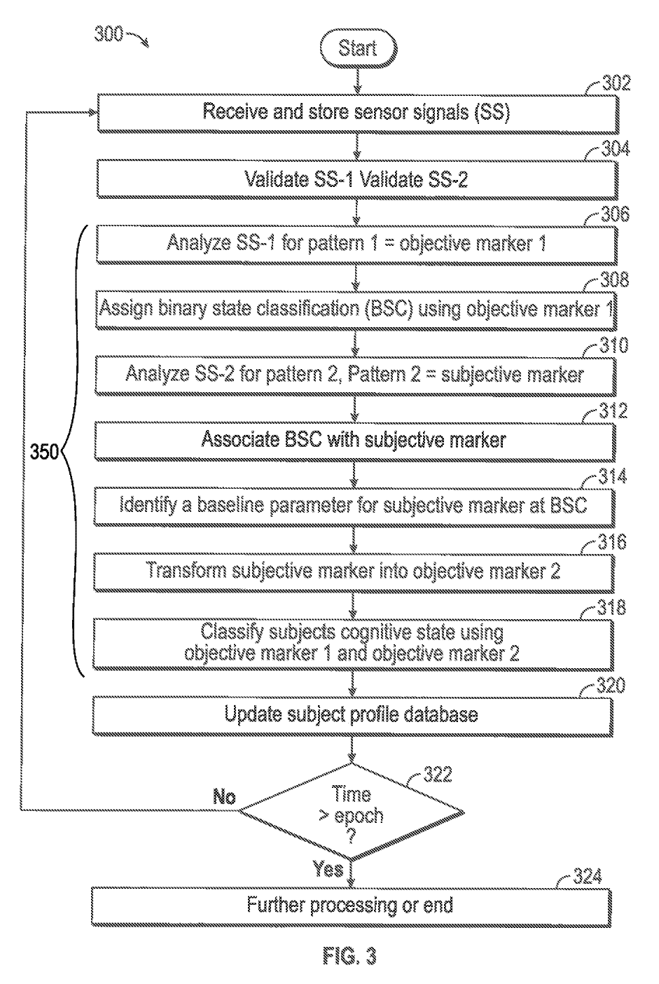

As mentioned, the system for assessment of cognitive state 102 may be used to implement a method 300, as shown in the flow charts of FIGS. 3-5. For illustrative purposes, the following description of method 300 may refer to elements mentioned above in connection with FIGS. 1-2. In practice, portions of method 300 may be performed by different components of the described system 102. It should be appreciated that method 300 may include any number of additional or alternative tasks, the tasks shown in FIGS. 3-5 need not be performed in the illustrated order, and method 300 may be incorporated into a more comprehensive procedure or method having additional functionality not described in detail herein. Moreover, one or more of the tasks shown in FIGS. 3-5 could be omitted from an embodiment of the method 300 as long as the intended overall functionality remains intact.

In order to start the method 300, the system for assessment of cognitive state 102 is initialized. Initialization generally comprises uploading or updating instructions and applications 160 as required for operation of the system for assessment of cognitive state 102. This may include: the executable program 162; contents of the database 156; and, any associated stored variables 164 (examples of variables for storage include: a first threshold, a second threshold, a segment size, etc.). Also part of initialization, a data storage location defined as a buffer 166 is initialized. At 302, the sensor signals 13 from a subject 10 are received and stored.

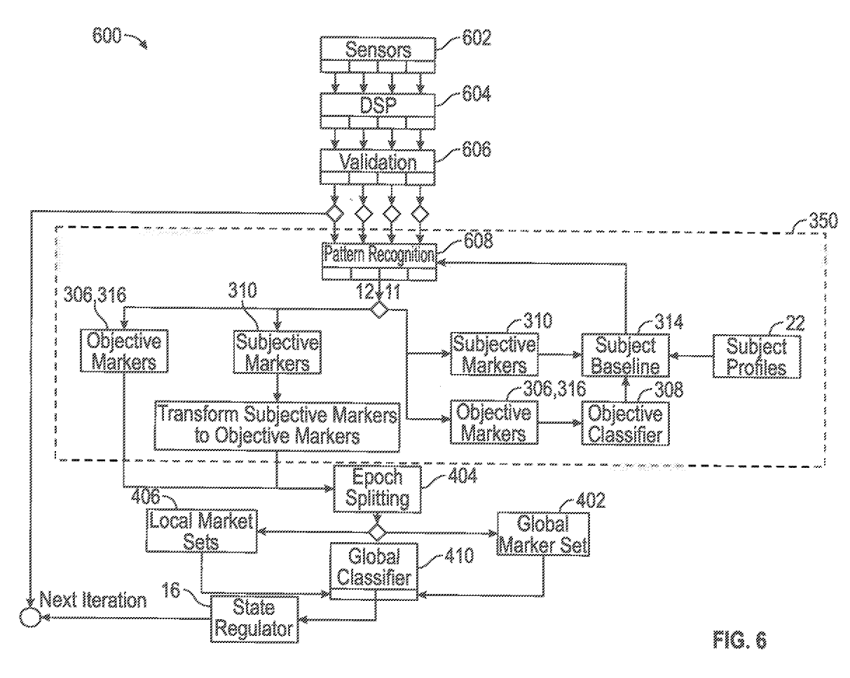

For the purposes of developing concepts employed by the method 300, a simple example that uses two contemporaneously received sensor signals (SS_1 and SS_2) is now provided. It may be further helpful to consider SS_1 to be an eyelid position signal and SS_2 to be electrical signals from the heart. It is to be appreciated that, in practice (and with specific reference to FIG. 6), SS_1 and SS_2 are two of a plurality of contemporaneously received sensor signals 13 representative of biometric data 11.

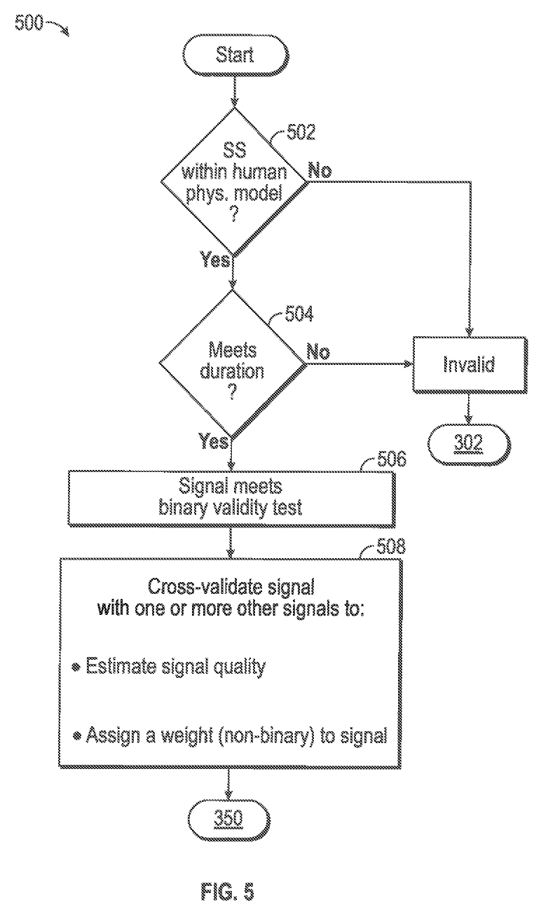

At 304 sensor signals are validated based on referencing human physiology models 20. For the example, at 304, the SS 1 is validated, and the SS 2 is validated. Method steps 500 of FIG. 5, described below, provide further detail for the validation step 304.

Steps 306-318 may be jointly referred to as employing an adaptive feedback filter (350). The adaptive feedback filter 350 steps, as applied to the present example, are as follows. At 306, SS_1 is analyzed to identify a pattern, pattern 1, therein. For this example, the pattern 1 is “the eyes are open.” In the example, the pattern 1 is an objective marker 1, in that it does not vary from subject to subject. At 308, a binary state classification (BSC) is assigned based on the objective marker 1. In this example, the BSC is “awake” based on “the eyes are open.”

At 310, SS_2 is analyzed to identify a pattern, pattern 2. In this example, the SS_2 is “heart rate,” which, in practice, will additionally have an associated number and units. As may be appreciated, heart rates for a given state vary from subject to subject, and therefore pattern 2 is a subjective marker. At 312, the BSC (awake) is associated with the subjective marker (heart rate). At 314, the subjective marker is further analyzed and a baseline parameter for the subjective marker (heart rate) is identified. At 316, using the baseline parameter and adaptive data filtration, the subjective marker (heart rate) is transformed into an objective marker, objective marker 2. Continuing with the example, at 316, the heart rate for the subject 10 is transformed into objective marker 2 (also, “awake”).

At 318, the method 300 classifies the cognitive state of the subject 10 (in this example, as “awake”) based on a combination of objective marker 1 and objective marker 2. At 320, the subject profile database for subject 10 may be updated to have an association between the specific heart rate baseline parameter from 314 and binary state classification from 312. As mentioned, method 300 cycles for a configurable, predetermined, amount of time, the epoch. At 322, if the epoch has elapsed, the method may end or proceed to further processing at 324. If the epoch has not elapsed, the method 300 may continue receiving and storing sensor signals at 302.

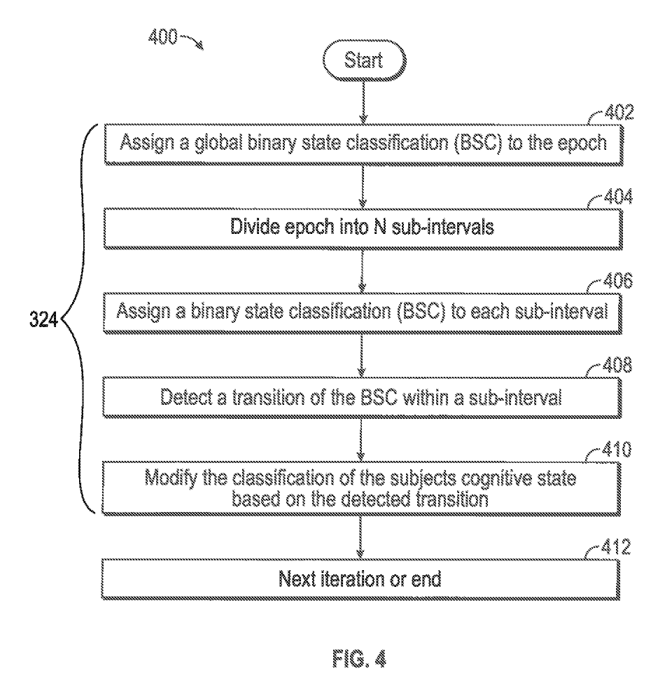

Method steps 400, in FIG. 4, detail one embodiment of further processing (324). At 402, a global binary state classification BSC is assigned to the epoch. In this example, it is determined that the subject 10 shall be classified as awake for the whole epoch. At 404, the epoch is divided into N sub-intervals. At 406, each of the N sub-intervals is assigned a unique respective binary state classification BSC. At this point, it's possible that not all of the sub-intervals have the same BSC. For example, with an N of 10, and assigning sub-interval 2 as “asleep” (in keeping with the binary concept, sub-interval 2 is actually assigned “not awake”). It is readily understood that for this to occur, there must be at least one transition between binary states within the epoch. For example, perhaps before sub-interval 2, or early in sub-interval 2, there's a transition from awake to not awake, and that BSC endures until near the end of sub-interval 2, or until just after sub-interval 2, at which time, there's a transition from “not awake” back to awake. At 408, the N sub-intervals are further processed, and a transition of the BSC is detected within its respective sub-interval. At 410, the classification of the subject's cognitive state may be modified based on the detected transition of the BSC. A conflict that arises when a binary state classification of a sub-interval does not agree with the binary state classification of the epoch, or when a detected transition in a sub-interval does not agree with the BSC assigned to the sub-interval. In some embodiments, a conflict may be resolved by the state regulator 16 before commands are generated for the user interface 18. In other embodiments, the control module 104 performs the conflict resolution.

As mentioned, validation step 304 may be further described by method steps 500. As used herein, “validation” may include two phases, the first being a binary validation step, and the second being a weighted cross-validation step. As described above, the first phase references the respective human physiology model 20, and determines whether SS_1 is valid, and whether SS_2 is valid, by comparing them individually to their respective human physiology models 20 (at 502). If a sensor signal is outside of the respective human physiology model 20, it is invalid. Further, if a sensor signal is within the human physiology model 20, then it may be additionally required to be so for a predetermined duration of time (504); if it does not meet the predetermined duration of time, it is invalid. When a sensor signal is invalid, the method may return to receiving sensor signals 13 (302). At 506, the sensor signal has met its binary validity test.

Subsequently, in a second phase, the weighted cross-validation step (508), SS_1 and SS_2 are cross-validated. I.e., SS_1 is used to estimate a signal quality of SS_2, and based on that, SS_2 is assigned a non-binary weight. Likewise, in the second phase, SS_2 is used to estimate a signal quality of SS_1, and based on that, SS_1 is assigned a non-binary weight. The first weight and the second weight may be the same, and they may be different. As used herein, the quality of the signal generally translates to a signal confidence, or the extent to which the signal “makes sense,” which means that it is consistent with the BSC determined thus far. The signal quality is indicated by a weight that reflects the signal confidence. As an example of this in practice, the eyelid position (SS_1) may be used to determine whether the heart electrical signal (SS_2) makes sense, and the heart electrical signal (SS_2) may be used to determine whether the eyelid position (SS_1) makes sense. For example, a heart electrical signal that generally represents anxiety is less likely to be contemporaneous with eyes closed. As a result of this phase of validation SS_1 and SS_2 each are assigned a weight. The control module's 104 cross-validity rules have a technical effect that the validity of one signal is determined from features of other signals. The control module's 104 cross-validity rules also have the technical effect of increasing efficiency near BSC transitions and thresholds, where error rates from simple noise removal and binary validation techniques are often the highest.

As mentioned, the flow chart of FIGS. 3-5 is a simplified example to illustrate processes and transformations performed by the system 102. In FIG. 6 the system 102 receives and processes a plurality of sensor signals 13 concurrently. When processing a plurality of contemporaneously received sensor signals concurrently in this system 102, many sensor signal issues can be addressed. For example, a camera detecting a subject's movement can be used to invalidate a touchscreen pressure signal indicating no movement, and a short term absence of a heart electrical signal can be reconstructed on condition that validity for that sensor signal has been determined to be high (i.e., have a large weight). Further, based on an assigned BSC, all sensor signal 13 features are used to update a subject specific baseline for a given subject 10. In addition, the database of subject profiles can be used for further study and analysis of variations between individual subjects.

Turning now to the flow of information shown in FIG. 6: Sensors 602 generate electrical signals which are signal processed at 604 to remove artifacts and noise. Validation of sensor signals 13 occurs at 606. The adaptive feedback filter 350 steps involve two iterations. For reference, in FIG. 6, there is an I1 label for first iteration events, and an I2 label for second iteration events. The first adaptive feedback filter 350 iteration includes: pattern recognition at 608 leads to identifying an objective marker (306), which leads to assigning a BSC at 308, and identifying a subject baseline parameter 314. In this manner, all patterns (markers), from all of the plurality of sensor signals contemporaneously received, are used to update the subject specific baseline at (314). The second adaptive feedback filter 350 iteration includes: pattern recognition (608) based on the previously determined subject baseline parameter (314) to identify a subjective marker (310) and transform that into objective marker 2 (316).

To summarize the control module's 104 adaptive feedback filter 350 functionality: Adaptive feedback filter 350 divides identified patterns into objective and subjective markers. The objective markers are processed in internal first iteration (I1) to yield an ‘objective’ state assessment. The result is fed back into the second iteration (I2). With these control module 104 adaptive feedback rules, instead of requiring a user entry to parametrize a generic model, when the control module 104 makes an objective determination, it may add the subject's current subjective markers to a distribution of that subjective marker that is associated with that objective state. For example, when the control module 104, on a first iteration determines that a subject is ‘objectively’ unlikely to be drowsy, a subjective marker, such as the subject's current heart rate, is added to a distribution of non-drowsy heart rates of the subject. The technical effect of these control module 104 adaptive feedback rules is a reduction in initial calibration of models and a reduction in the need for user interaction with the system 102. After the second iteration is completed, objective marker 1 and objective marker 2 (transformed subjective marker 1) concurrently proceed to epoch splitting 404.

Epoch splitting 404 produces the N sub-intervals described above. The global marker set 402 is the one or more BSCs assigned to the entire epoch. Local markers 406 are the BSCs that are assigned to the N individual sub-intervals. Finally, the epoch BSC and N-subset BSCs are processed by the system 102. Expert rules in program 162 combine results of all available sensor signals 13 using their respective determined weights (determined during validation 304) and decide the final binary cognitive state (i.e., modify the BSC if necessary) representing the entire epoch.

Accordingly, the exemplary embodiments described above provide a technologically improved system for assessment of cognitive state 102. The per-signal classification (a technical effect of the epoch splitting and classification) and fusion of its results (a technical effect of the conflict resolution and BSC modification) combined with the technical effect of the employed binary and weighted signal validity methodology results in a more robust cognitive state detection and classification system 102. This technologically enhanced system 102 is of particular value in complex working environments, and in scenarios in which some of the sensor signals are temporarily unavailable.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for classifying a subject's cognitive state, the method comprising:
at a control module, for a predetermined amount of time (epoch), continuously:
receiving and storing sensor signals comprising a first sensor signal (SS_1) from a first sensor configured to sense a first aspect of the subject, and a second sensor signal (SS_2) from a second sensor configured to sense a second aspect of the subject;
determining that SS_1 is valid, based on human physiology models;
determining that SS_2 is valid, based on the human physiology models;
analyzing SS_1 to identify a pattern therein, the pattern defined as an objective marker; and
sequentially performing the steps of,
(a) assigning a binary state classification based on the objective marker; and
(b) analyzing SS_2 to identify a pattern therein, the pattern defined as a subjective marker;
(c) creating an association between the binary state classification and the subjective marker;
(d) identifying a baseline parameter for the subjective marker, the baseline parameter being unique for the subject;
(e) transforming the subjective marker to a second objective marker using adaptive data filtration and the baseline parameter; and
(f) classifying the subject's cognitive state using the first objective marker and the second objective marker.

2. The method of claim 1, further comprising:
processing the sensor signals to assign a global binary state classification to the epoch; dividing the epoch into N sub-intervals; and
assigning a binary state classification to each of the N sub-intervals.

3. The method of claim 2, further comprising,
resolving a conflict between a binary state classification of a sub-interval with the binary state classification of the epoch.

4. The method of claim 1, wherein:
determining that SS_1 is valid is further based on processing SS_1 with a predetermined duration of time; and
determining that SS_2 is valid is further based on processing SS_2 with the predetermined duration of time.

5. The method of claim 4, wherein:
determining that SS_1 is valid is further based on cross-validating SS_1 with SS 2;
determining that SS_2 is valid is further based on the cross-validating of SS_1 with SS_2; and further comprising:
based on the cross validating of SS_1 with SS_2, assigning (i) a first weight to SS_1, and (ii) a second weight to SS_2.

6. The method of claim 5, wherein assigning a binary state classification is based on the first weight and the second weight.

7. The method of claim 6, further comprising:
detecting, within one of the sub-intervals, a transition of the binary state classification; and
modifying the classification of the subject's cognitive state based on the detected transition of the binary state classification in the sub-interval.

8. A system for assessment of cognitive state of a subject, comprising:
a source of sensor signals associated with the subject;
a state regulator configured to receive a binary cognitive state and to generate therefrom commands for a user interface; and
a control module comprising human physiology models, the control module configured to:
receive a first sensor signal (SS_1) and a second sensor signal (SS_2);
determine that SS_1 is valid with a binary validity test;
determine that SS_2 is valid with a binary validity test;
analyze SS_1 to identify a pattern therein, the pattern defined as an objective marker; and
sequentially perform the steps of,
(a) assign a binary state classification based on the objective marker; and
(b) analyze SS_2 to identify a pattern therein, the pattern defined as a subjective marker;
(c) create an association between the binary state classification and the subjective marker;
(d) identify a baseline parameter for the subjective marker, the baseline parameter being unique for the subject;
(e) transform the subjective marker to a second objective marker using adaptive data filtration and the baseline parameter; and
(f) classify the subject's cognitive state using the first objective marker and the second objective marker.

9. The system of claim 8, wherein the control module is further configured to:
process the sensor signals to assign a global binary state classification to the epoch; dividing the epoch into N sub-intervals; and
assign a binary state classification to each of the N sub-intervals.

10. The system of claim 9, wherein the control module is further configured to:
resolve a conflict between a binary state classification of a sub-interval with the binary state classification of the epoch.

11. The system of claim 8, wherein the control module is further configured to:
determine that SS_1 is valid further based on processing SS_1 with a predetermined duration of time; and
determine that SS_2 is valid further based on processing SS_2 with the predetermined duration of time.

12. The system of claim 11, wherein the control module is further configured to:
determine that SS_1 is valid further based on cross-validating SS_1 with SS_2;
determine that SS_2 is valid further based on the cross-validating of SS_1 with SS_2; and further comprising:
based on the cross validating of SS_1 with SS_2, assign (i) a first weight to SS_1, and (ii) a second weight to SS_2.

13. The system of claim 12, wherein the control module is further configured to assign a binary state classification based on the first weight and the second weight.

14. The system of claim 10, wherein the control module is further configured to:
detect, within the sub-intervals, a transition of the binary state classification; and
modify the classification of the subject's cognitive state based on the detected transition of the binary state classification.

15. The system of claim 14, further comprising a user interface operatively coupled to the state regulator, and configured to generate cognitive state mitigating feedback responsive to commands from the state regulator.

16. A method for classifying a subject's cognitive state, the method comprising:
at a control module, continuously:
receiving and storing sensor signals comprising a first sensor signal (SS_1) from a first sensor configured to sense a first aspect of the subject, and a second sensor signal (SS_2) from a second sensor configured to sense a second aspect of the subject;
analyzing SS_1 to identify a pattern therein, the pattern defined as an objective marker;
assigning a binary state classification based on the objective marker;
analyzing SS_2 to identify a pattern therein, the pattern defined as a subjective marker;
cross validating SS_1 with SS_2 to thereby (1) determine that SS_1 is valid, (2) determine that SS_2 is valid, and (3) assign (i) a first weight to SS_1, and (ii) a second weight to SS_2;
creating an association between the binary state classification and the subjective marker;
identifying a baseline parameter for the subjective marker, the baseline parameter being unique for the subject;
transforming the subjective marker to a second objective marker using adaptive data filtration and the baseline parameter; and
classifying the subject's cognitive state using the first objective marker and the second objective marker.

17. The method of claim 16, further comprising:
processing the sensor signals for a period of time defined as an epoch; and
assigning a global binary state classification to the epoch;

18. The method of claim 17, further comprising:
dividing the epoch into N sub-intervals; and
processing the N sub-intervals to assign a unique binary state classification to each of the N sub-intervals.

19. The method of claim 18, wherein assigning a binary state classification is based on the first weight and the second weight.

20. The method of claim 19, further comprising,
identifying a conflict between a binary state classification of a sub-interval with the binary state classification of the epoch; and
resolving the conflict between a binary state classification of a sub-interval with the binary state classification of the epoch.
